(19) United States
(12) Patent Application Publication
(10) Pub. No.: US 2026/0104371 A1
(43) Pub. Date: Apr. 16, 2026

(54) METHOD FOR EXTRACTING DISCRETE NEUTRON COMPONENTS FROM PHOTO-NUCLEAR REACTIONS USING A TRAINED NEURAL NETWORK

(71) Applicant: COMMISSARIAT A L'ENERGIE ATOMIQUE ET AUX ENERGIES ALTERNATIVES, Paris (FR)

(72) Inventors: Clément BESNARD VAUTERIN, Gif-sur-Yvette Cedex (FR); Valentin BLIDEANU, Gif-sur-Yvette Cedex (FR); Benjamin RAPP, Gif-sur-Yvette Cedex (FR)

(73) Assignee: COMMISSARIAT A L'ENERGIE ATOMIQUE ET AUX ENERGIES ALTERNATIVES, Paris (FR)

(21) Appl. No.: 19/115,004

(22) PCT Filed: Sep. 28, 2023
(86) PCT No.: PCT/FR2023/051502

§ 371 Date: Mar. 25, 2025

(30) Foreign Application Priority Data
Sep. 30, 2022 (FR) FR2210022

(51) Int. Cl. G01N 23/222; G16C 20/10; G16C 20/70

(52) U.S. Cl. CPC G01N 23/222; G16C 20/10; G16C 20/70

(57) ABSTRACT

The invention relates to a method for extracting discrete neutron components (from photo-nuclear reactions between photons and at least one chemical element to be detected) of a neutron spectrum from photo-nuclear reactions obtained by irradiation, of a material comprising said at least one chemical element to be detected, with a photon source, at least one energy of the photons of the source being greater than the photo-nuclear reaction threshold of the chemical element to be detected, using a multilayer and multichannel neural network having an architecture with a convolution stage and a deconvolution stage. The method comprises a preliminary step of training the neural network by supervised learning and a prediction step, using the trained neural network.

### TECHNICAL FIELD

The technical field of the invention is that of the processing of neutron spectra using a neural network to extract useful information from them, for example in the context of the detection of hazardous, toxic or illicit substances contained in an object.

### PRIOR ART

It is known that neutron and photon radiation sources can be used to detect, from specific nuclear reactions, the presence of certain light elements (namely nitrogen, carbon, oxygen, chlorine) in various materials. The presence can thus be detected of hazardous, toxic or illicit substances such as explosives, certain toxic gases (toxic gases containing chlorine) or drugs, contained or hidden in an object to be probed (which can, for example, be of the container, package or suitcase type).

Among the known detection methods, mention can be made of the active neutron interrogation (ANI) method, the so-called “tagged” photon method and the active photon interrogation (API) method.

The active neutral interrogation (ANI) method uses neutron-induced reactions. These reactions are used to detect light elements (C, N, O, Cl) in materials using the associated particle technique (APT) in particular. This detection is based on measuring the gamma radiation produced by these reactions. However, accurate measurement of gamma spectra is generally difficult considering the experimental conditions, frequently characterised by substantial background noise. Indeed, gamma radiation is largely present, because it can be produced readily by a multitude of mechanisms (reactions with other chemical elements than the chemical elements of interest, radioactive decay of the radioisotopes created by activation or which are naturally present in the materials, etc.), which results in complex spectra making separation of the different contributions of the chemical elements difficult.

The so-called “tagged” photon technique is based on detecting neutrons coincident with the electrons from a bremsstrahlung radiation source. This technique allows neutron component extraction for each bremsstrahlung photon energy. However, it requires the set-up of complex methods with specialised high-frequency electronics for detecting coincident particles, and heavy, restrictive and costly equipment, in particular magnetic dipoles which are not really compatible with the development of a compact detection device in the case of a domestic security application.

Finally, the active photon interrogation (API) method is a detection based on the use of photon sources. It uses photo-nuclear reactions, the effective cross-sections (probabilities) whereof are well known for most of the nuclei of light elements such as nitrogen, carbon, oxygen, chlorine.

Photo-nuclear reactions have the specificity of being carried out at a threshold, i.e. they can only take place if the initial photons have an energy greater than a certain value, which is specific to each nucleus.

In the case of nitrogen, for example, which is detected using the neutrons produced by photo-nuclear reactions (γ,n), the minimum photon energy must be greater than 10 MeV.

In the case of the detection of chlorine by irradiation with photons using the API method, current techniques are based on creating, via photo-nuclear reactions (γ,n), radioisotopes, the decay whereof causes gamma radiation emission. Spectrometry measurement of this gamma radiation makes it possible to identify these radioisotopes and thus go back to the elements causing their production. However, the API method coupled with gamma spectrometry is very complex to set up given the scale of the gamma noise generated by numerous mechanisms.

The photons used in active photon interrogation therefore cannot be obtained with conventional methods based on radioactive sources, because they do not allow sufficient energy levels to be reached. It is therefore necessary to have sufficiently intense high-energy photon sources to obtain a satisfactory signal-to-noise ratio. Moreover, the source must furthermore be as compact as possible to be deployed in the field, in the case of an application to illicit substance detection, for example.

Traditionally, the high-energy photons (greater than or equal to 6 MeV) used to implement the API method are bremsstrahlung photons, which are produced using an electron accelerator. In a known manner, in an electron accelerator, an electron beam of sufficient energy (several MeV) strikes a target consisting of a material of high atomic number Z, which gives rise to braking radiation (bremsstrahlung photons). The disadvantage of using an electron accelerator lies in that the photon source thus obtained (bremsstrahlung photons) is characterised by a continuous energy spectrum. However, only a few percent of the photons obtained with this method (i.e. those having an energy greater than 6 MeV necessary to induce photofission reactions (γ,f)) can be used, the large majority of the photons causing unnecessary irradiation of the substances present in the object to be probed.

Ideally, such a source should be based on the use of single-energy photons obtained, for example, from a low-energy photon source, in order to be able to identify specific signatures (or fingerprints) composed of discrete neutron components in the spectra of the neutrons emitted in reactions (γ,n) induced by these photons on light nuclei (C, N, O, Cl). Discrete neutron components will be the signature of the presence of certain light elements; the position of these discrete neutron components will inform us on the nature of the light elements in question and the height of these discrete neutron components will inform us on their relative quantity.

However, the inventors'previous research aimed at studying the set-up of a single-energy photon source showed numerous limitations linked, in particular, with the intensity that such a source could have and its isotropy.

Within the scope of the present invention, the inventors focussed on API detection and sought to design a method for processing neutron spectra from photo-nuclear reactions where several photon energies are at play. In other words, the inventors sought to process neutral spectra obtained by irradiation using bremsstrahlung photons or photons with several lines (therefore at least two lines).

### DISCLOSURE OF THE INVENTION

This aim is particularly achieved thanks to the use of a trained neural network.

The invention thus relates to a method for extracting discrete neutron components, from photo-nuclear reactions between photons and at least one chemical element to be detected, of a neutron spectrum from photo-nuclear reactions obtained by irradiation, of a material comprising said at least one chemical element to be detected, with a photon source, at least one energy of the photons of the source being greater than the photo-nuclear reaction threshold of said chemical element to be detected, using a multilayer and multichannel neural network having an architecture with a convolution stage and a deconvolution stage;
wherein, if said at least one chemical element to be detected is chosen from carbon, nitrogen, oxygen or chlorine and if the photon source is a first multi-energy photon source, the method comprises:
   a preliminary step of training the neural network by supervised learning, which is performed by iteration using:
      first neutron spectra, from photo-nuclear reactions obtained by irradiating the material with bremsstrahlung photons; and
      second neutron spectra, each of the second neutron spectra being from photo-nuclear reactions induced by irradiating the material with photons of a single energy, which is greater than the photo-nuclear reaction threshold of the chemical element to be detected, and less than or equal to the maximum energy of the photons of said first multi-energy photon source,
the first and second spectra being notional spectra established by a Monte-Carlo simulation, the first spectra being supplied at the input of the neural network, and the second spectra serving to compute a cost function intended to adjust the weights and biases of the neural network; and
   a prediction step, using the trained neural network, comprising:
      supplying, at the input of the trained neural network, the neutron spectrum from photo-nuclear reactions induced by irradiating the material with said first photon source; and
      if the irradiated material includes, in its composition, at least one chemical element chosen from carbon, nitrogen, oxygen, or chlorine, obtaining, at the output of the trained neural network and for at least one specific energy of interest of said first photon source, a predicted neutron spectrum, including discrete neutron components from photo-nuclear reactions of said chosen chemical element with said specific energy of interest; or
wherein, if the material to be irradiated comprises said at least one chemical element to be detected chosen from carbon, nitrogen, oxygen or chlorine, and at least one other different chemical element, capable of emitting neutrons and which is not carbon, nitrogen, oxygen or chlorine, and if the photon source is a second single-energy or multi-energy (line or bremsstrahlung) photon source, the method comprises:
   a preliminary step of training the neural network by supervised learning, which is performed by iteration using:
      first neutron spectra, from photo-nuclear reactions obtained by irradiating the material with bremsstrahlung, line or single-energy photons; and
      second neutron spectra, each of the second neutron spectra being from photo-nuclear reactions induced by irradiating the material with photons of an energy greater than the photo-nuclear reaction threshold of the chemical element to be detected and greater than the photo-nuclear reaction threshold of said other different chemical element;
the first and second spectra being notional spectra established by a Monte-Carlo simulation, the first spectra being supplied at the input of the neural network, and the second spectra serving to compute a cost function intended to adjust the weights and biases of the neural network; and
   a prediction step, using the trained neural network, comprising:
      supplying, at the input of the trained neural network, the neutron spectrum from photo-nuclear reactions induced by irradiating the material with said second photon source; and
      if the irradiated material includes, in its composition, at least one chemical element chosen from carbon, nitrogen, oxygen or chlorine, obtaining, at the output of the trained neural network and for at least one specific energy of interest of said second photon source, a predicted neutron spectrum, including discrete neutron components from photo-nuclear reactions of said chemical element to be detected with said specific energy of interest.

In the first scenario where the at least one chemical element to be detected is chosen from carbon, nitrogen, oxygen or chlorine and if the photon source is a first multi-energy photon source (hereinafter referred to as “first scenario”), then it is possible to extract, from a neutron spectrum produced by a bremsstrahlung photon or line photon source, discrete neutron components, as if the neutron spectrum had been produced by irradiation with a single-energy photon source (i.e. of only one energy).

In the second scenario where the material to be irradiated comprises said at least one chemical element to be detected chosen from carbon, nitrogen, oxygen or chlorine, and at least one other different chemical element, capable of emitting neutrons and which is not carbon, nitrogen, oxygen or chlorine, and if the photon source is a second single-energy or multi-energy (line or bremsstrahlung) photon source (hereinafter referred to as “second scenario”), it is possible to extract, from a neutron spectrum produced by irradiating a complex assembly (material comprising at least one light element and object wherein it is located) using a photon source (whether the photons are bremsstrahlung, multi-line or single-energy), discrete neutron components created by said at least one light element.

In these two scenarios, only the training step is different. The spectra provided for computing the cost function are neutron spectra from single-energy irradiation in the first scenario, and spectra from irradiation on single elements in the second scenario. “Single element” means that the irradiated material consists of only one element (or, in other words, a pure element), that is to say that, in the material, the element is not mixed with other different elements.

By way of illustration, in the second scenario, during the training step, the neural network is supplied with neutron spectra from irradiation with bremsstrahlung photons of samples or various substances at the network input. Learning, for neutral networks, consists of computing parameters in such a way that the outputs of the neural network are, for the examples used during learning, as close as possible to the “desired” outputs (here: neutron spectra from irradiation with single-energy photons). During learning, it is therefore sought to minimise the deviation between the actual responses of the network and the “desired” responses, by modifying the parameters via successive steps. The network will then be “shown” these “desired” responses for each neutron spectrum from irradiation with bremsstrahlung photons of samples or various substances, and supplied at the network input. These “desired” responses do not actually represent input data per se, like neutron spectra from irradiation with bremsstrahlung photons.

According to an advantageous alternative of the invention, the specific energy of interest is greater than or equal to 17 MeV.

In fact, in the prediction step, a neutron spectrum can be obtained for a specific energy of interest or several spectra, for two or more different source energies. However, in order to restrict computing costs, it is preferred to predict a neutron spectrum for a single source photon energy, chosen according to the chemical element to be detected. Within the scope of application to explosive detection where the detection of nitrogen is of interest, the choice is made of 17 MeV energy, which is the most suitable for nitrogen detection.

The invention also relates to a method for detecting a chemical element to be detected chosen from carbon, nitrogen, oxygen or chlorine, the method comprising:
irradiating, with a multi-energy photon source (the photons can therefore be bremsstrahlung or line photons), a material comprising said chemical element;
detecting the neutrons emitted by the irradiated material and acquiring a corresponding neutron spectrum;
extracting, from the neutron spectrum acquired, discrete neutron components due to said chemical element to be detected, by implementing the method for extracting discrete neutron components as disclosed hereinabove, in the scenario where the photon source is the first source;
comparing the discrete neutron components extracted with a library of discrete neutron components associated with carbon, nitrogen, oxygen and chlorine, and identifying a correlation, whereby the presence of said chemical element is inferred.

The invention finally relates to a method for detecting a material to be detected contained in an object to be probed, the material to be detected comprising at least one chemical element to be detected chosen from carbon, nitrogen, oxygen or chlorine, and the object to be probed comprising at least one other different chemical element, capable of emitting neutrons and which is not carbon, nitrogen, oxygen or chlorine, the method comprising:
irradiating, with a photon source (which can be a bremsstrahlung, line or single-energy photon source), the object to be probed and the material to be detected contained therein;
detecting the neutrons emitted by the object to be probed and the material to be detected and acquiring a corresponding neutron spectrum;
extracting, from the neutron spectrum acquired, discrete neutron components due to said at least one chemical element, by implementing the method for extracting discrete neutron components as disclosed hereinabove in the scenario where the photon source is the second source;
comparing the discrete neutron components extracted with a library of discrete neutron components associated with materials comprising said at least one first chemical element, and identifying a correlation, whereby the presence of said material to be detected is inferred.

In the various aspects of the invention, the use of a neural network makes it possible to avoid the need for a single-energy source in favour of the use of a more conventional, more intense and readily accessible source such as, for example, a source based on a linear electron accelerator generating a continuous energy spectrum of photons (bremsstrahlung radiation) which have the advantage of being substantially forward-oriented (giving a directional source).

Within the scope of the invention, reference is made to neutron compounds which are described as “discrete”. This is understood to mean that the neutron components form structures in peaks of varying widths, which are clearly outside the continuum of the neutron spectra. The full width at half-maximum of these peaks will depend in particular on the energy resolution of the spectra.

### BRIEF DESCRIPTION OF THE DRAWINGS

Other aspects, aims, advantages and features of the invention will become more apparent upon reading the following detailed description of preferred embodiments thereof, given as a non-limiting example, and made with reference to the appended drawings, wherein:

FIG. 1 schematically represents a set-up to illustrate an embodiment of the invention;

FIGS. 2a to 2c respectively represent a bremsstrahlung photon spectrum, as obtained after the target 2 of FIG. 1, a spectrum of the neutrons produced by irradiating an explosive material with bremsstrahlung photons (FIG. 2b), as obtained by the detector 5 of FIG. 1, and a predicted neutron spectrum for an irradiation of the same explosive material with 17 MeV photons (FIG. 2c), as obtained at the output of a trained neural network 6 according to the invention, with an identification of the light elements present in its composition;

FIGS. 3a and 3b schematically represent a multilayer and multichannel neural network (FIG. 3a) and a diagram to describe an architecture of this neural network, with a convolution stage and a deconvolution stage (FIG. 3b);

FIG. 4 represents a triangle of the standardised nitrogen, carbon and oxygen proportions of several substances or products, with an identification of the groups under the illicit substance category.

### DETAILED DISCLOSURE OF SPECIFIC EMBODIMENTS

According to a first aspect of the invention, the method according to the invention makes it possible to extract neutron components from photo-nuclear reactions, for each photon energy making up a source, the photons whereof have a continuous energy (bremsstrahlung) or multi-energy (line) spectrum. The method according to the invention makes it possible to replace the existing so-called “tagged” photon method by the use of a multilayer and multichannel neural network, which makes it possible to avoid the need for specialised electronics and magnetic dipoles required by the “tagged” photon method. The method according to the invention also makes it possible to avoid the need to use a single-energy source. Neutron spectra are obtained here for each photon energy in post-processing, without additional physical resources than those conventionally used in API detection, thus limiting the costs and size of the light element detection device used.

Thus, the use of a so-called “neural network” deep learning network according to the invention makes it possible to retrieve the neutron contributions in the total spectrum generated by photons from a bremsstrahlung or line source for the different photon energies of the source. Identification of the light elements is thus enabled in the different neutron contributions extracted.

According to a second aspect of the invention, the method according to the invention also makes it possible to extract the neutron components due to light elements of a complex matrix (which can be the material wherein the light element(s) are found and/or the object wherein the material is located).

Regardless of the aspects of the invention, the specificity of the proposed approach consists of learning and use of a multilayer and multichannel neural network trained in such a way as to be able to predict the neutron components for each bremsstrahlung or line photon energy, when it is supplied with neutron spectrum from irradiating materials with bremsstrahlung or line photons, or when it is sought to analyse neutron spectra from any type of photon source (single-energy, line or bremsstrahlung) to extract the neutron contributions of the light elements of a complex matrix.

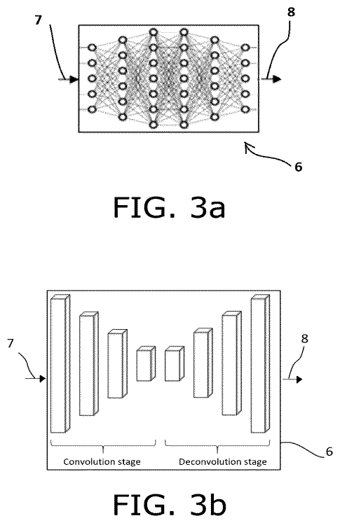

The neural network 6 is multilayer and multichannel (FIG. 3a).

It is multilayer, because it has an input layer, an output layer and one or more intermediate layers, called hidden layers.

It is multichannel, because each layer includes several channels. Each channel will represent an energy interval of the spectrum. Each energy interval of a neutron spectrum corresponds to an input node 7 and to an output node 8. For example, if it is sought to retrace a neutron spectrum with an energy ranging from 1 to 11 MeV, it is possible to have 100 channels for each energy with an increment (sampling interval) of 0.1 MeV. The higher the number of channels, the lower the value of the sampling interval and the more precise the resolution obtained.

The neutral network 6 has a two-part architecture. In fact, it is composed of a convolution stage and a deconvolution stage (FIG. 3b).

The first stage allows the extraction of characteristics and patterns of the neutron spectra introduced at the input 7 of the network 6 and allows data reduction. The second stage performs the reconstruction of a spectrum using the characteristics extracted by the first stage, while retrieving the dimension of the initial data.

In a known manner, supervised neutral network learning is based on the series of following steps, for n input-target value pairs, where n is an integer depending on the energy resolution of the spectra and corresponding to the number of energy intervals of the spectra:
1—presenting one of the “input spectrum—target spectrum” pairs (hereinafter “input-target spectra”) of the n intervals to the network;
2—computing a network prediction for the expected target; 3-using a cost function to compute the difference between the network prediction (output) and the target spectrum;
4—using the neural network learning algorithm to adjust the weights and biases of the network, such that the network produces better predictions on each presentation of an input-target pair.

It should be noted that steps 1 to 4 represent a single iteration (or learning cycle). Steps 1 to 4 are repeated for a certain number of iterations until the network starts producing sufficiently reliable results (i.e. outputs that are close enough to the targets considering the input values). The number of iterations required to train the neural network is not known initially, but it is generally greater than several hundreds, or thousands of times.

In this case, for each pair of input-target spectra, the input is a first notional spectrum (with reference to the name “first neutron spectra” used to define the invention), for example a neutron spectrum produced by bremsstrahlung photons and, for the target, a second notional spectrum (with reference to the name “second neutron spectra” used to define the invention) produced by a single-energy source. Thus, by way of example, supervised neural network learning is performed by iteration using neutron spectra from irradiation with bremsstrahlung photons (“first notional spectra”) which are supplied at the input 7 of the network and neutron spectra produced by single-energy photons (“second notional spectra”), which serve to compute a cost function which will allow the adjustment of the weights and biases of the network 6 so that its prediction is as close as possible to the spectra from the expected deconvolution.

For learning, notional spectra defined by Monte-Carlo simulation are used, which makes it possible to obtain a multitude of input-target pair data readily. Data augmentation techniques are applied to multiply the spectra by varying the intensity of each channel slightly and randomly in order to obtain a sufficient number of different spectra for network learning. For example, the Monte-Carlo N-particle (MCNP™) transport code, which makes it possible to model nuclear physics processes using the Monte-Carlo method, can be used.

Once its learning is complete, the weights and biases of the network thus adjusted are recorded and the neural network can be used for prediction purposes without a target spectrum being supplied to it.

For example, a neutron spectrum obtained by irradiation with a multi-energy photon source is supplied at the input of the trained network, which will deconvolve it and numerically extract its neutron components, for a specific energy of interest (it is also possible to extract the neutron components for several source energies, or for each source photon energy).

The analysis of the predicted neutron spectra obtained at the output of the neural network makes it possible, for example, to detect in a material irradiated with bremsstrahlung photons, the presence of light elements using peaks which become much more readily identifiable and for which they are the signature. Each peak in the predicted spectrum corresponds to an energy level of a residual nucleus produced by reaction (γ,n), which is therefore an isotope of the initial nucleus. The energy levels specific to each residual nucleus make it possible to identify the element present in the material to be detected 4.

To illustrate the invention, we will implement the method according to the invention using a bremsstrahlung photon source to detect TNT (of formula C₇H₅N₃O₆) contained in an object made of wood (cellulose, of formula (C₆H₁₀O₅)n) by inducing photo-nuclear reactions therein.

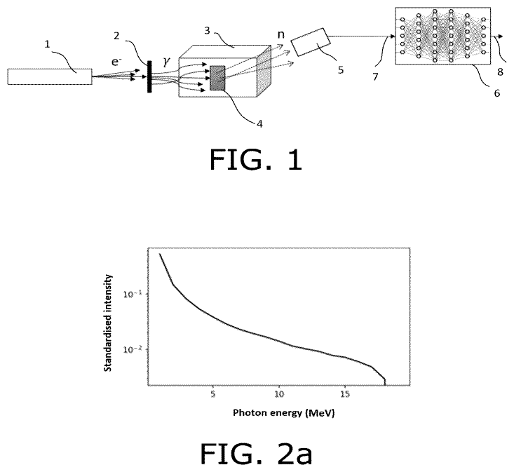

With reference to FIG. 1, an example of a set-up for carrying out the invention is illustrated schematically. A linear electron accelerator 1 generates radiation of electrons (e−) which are aimed at a specific target 2 (for example a thin gold target), which produces radiation of photons (γ) which are aimed at the object to be probed 3 and at the material to be detected 4 contained therein. The interaction of the photons γ with the material to be detected 4 produces neutrons, which are detected by a detector 5 for carrying out neutron spectrometry. The detector 5 can for example be chosen from Bonner sphere spectrometers or scintillators.

The neutron spectrum obtained at the output of the detector 5 is introduced, at the input 7, into the trained neural network 6, and, at the output 8 of this neural network, a predicted neutron spectrum is obtained, wherein the extracted discrete neutron compositions are present.

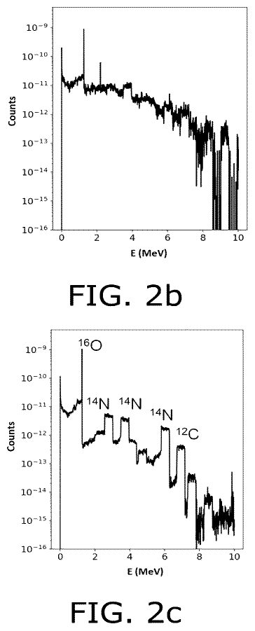

By way of example, the object to be probed 3 can be a package made of wood or cardboard containing, as material to be detected 4, a substance which is an explosive material, for example TNT; the bremsstrahlung photon spectrum obtained after the target 2 is illustrated in FIG. 2a; the neutron spectrum obtained at the output of the detector 5 by irradiating the explosive material with the bremsstrahlung photons is illustrated in FIG. 2b; the neutron spectrum predicted by the trained neural network 6 (and which is obtained at the output 8 of the neural network 6) is illustrated in FIG. 2c. The predicted spectrum is in fact identical to a spectrum obtained for an irradiation of the explosive material with photons of 17 MeV, which is a particularly advantageous energy in the context of nitrogen (N) detection for illicit substances; the different peaks (which correspond to the discrete neutron components extracted with the method) have been identified and attributed to the light elements ¹⁶O, ¹⁴N and ¹²C.

The position and height of the peaks forms a specific signature which is like a fingerprint and makes it possible to identify the presence of TNT. It is therefore possible to determine whether the package contains TNT.

In this example of implementation of the invention, a conventional source producing bremsstrahlung photons is used to induce photo-nuclear reactions in the material to be probed 3, but a single-energy or line source could have been used.

The invention is applicable in the field of detection with the active photon interrogation (API) method of illicit substances in containers and packages.

The detection of light elements can be carried out on the basis of the specific signatures in the neutron spectra extracted by the neural network.

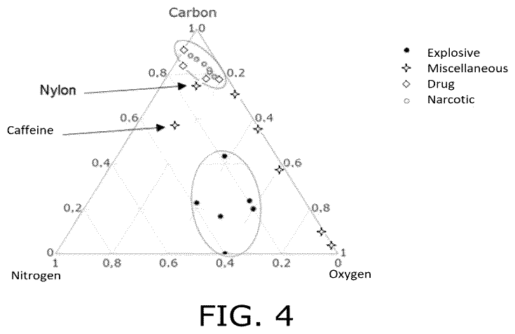

It is also possible to determine the concentrations of light elements such as nitrogen, carbon, oxygen or chlorine using the height of the discrete neutron components of these neutron spectra. As shown in FIG. 4, knowing the proportion of these light elements (C, N, O, Cl) effectively makes it possible to know the nature of the product or substance concealed in the probed object (package, container or other).

Nitrogen, for example is present in most explosives and may serve as a marker to detect an explosive trinitrotoluene (TNT, of chemical formula C₇H₅O₆N₃), cyclotrimethylenetrinitramine (RDX) material or any other conventional explosive, such as for example nitroglycerin (C₃H₅O₉N₃), or C4 (C₄H₆O₆N₆).

It is also possible to detect cocaine (C₁₇H₂₁NO₄).

Regarding toxic gases, their presence can be suspected with the detection of chlorine, present for example in phosgene (COCl₂).

1. Method for extracting discrete neutron components, from photo-nuclear reactions between photons and at least one chemical element to be detected, of a neutron spectrum from photo-nuclear reactions obtained by irradiation, of a material comprising said at least one chemical element to be detected, with a photon source, at least one energy of the photons of the source being greater than the photo-nuclear reaction threshold of said chemical element to be detected, using a multilayer and multichannel neural network having an architecture with a convolution stage and a deconvolution stage;
wherein, if said at least one chemical element to be detected is chosen from carbon, nitrogen, oxygen or chlorine and if the photon source is a first multi-energy photon source, the method comprises:
a preliminary step of training the neural network by supervised learning, which is performed by iteration using:
first neutron spectra, from photo-nuclear reactions obtained by irradiating the material with bremsstrahlung photons; and
second neutron spectra, each of the second neutron spectra being from photo-nuclear reactions induced by irradiating the material with photons of a same energy, which is greater than the photo-nuclear reaction threshold of the chemical element to be detected, and less than or equal to the maximum energy of the photons of said first multi-energy photon source,
the first and second spectra being notional spectra established by a Monte-Carlo simulation, the first spectra being supplied at the input of the neural network, and the second spectra serving to compute a cost function intended to adjust the weights and biases of the neural network; and
a prediction step, using the trained neural network, comprising:
supplying, at the input of the trained neural network, the neutron spectrum from photo-nuclear reactions induced by irradiating the material with said first photon source; and
if the irradiated material includes, in its composition, at least one chemical element chosen from carbon, nitrogen, oxygen, or chlorine, obtaining, at the output of the trained neural network and for at least one specific energy of interest of said first photon source, a predicted neutron spectrum, including discrete neutron components from photo-nuclear reactions of said chosen chemical element with said specific energy of interest; or
wherein, if the material to be irradiated comprises said at least one chemical element to be detected chosen from carbon, nitrogen, oxygen or chlorine, and at least one other different chemical element, capable of emitting neutrons and which is not carbon, nitrogen, oxygen or chlorine, and if the photon source is a second, single-energy or multi-energy photon source, the method comprises:
a preliminary step of training the neural network by supervised learning, which is performed by iteration using:
first neutron spectra, from photo-nuclear reactions obtained by irradiating the material with bremsstrahlung, line or single-energy photons; and
second neutron spectra, each of the second neutron spectra being from photo-nuclear reactions induced by irradiating the material with photons of an energy greater than the photo-nuclear reaction threshold of the chemical element to be detected and greater than the photo-nuclear reaction threshold of said other different chemical element;
the first and second spectra being notional spectra established by a Monte-Carlo simulation, the first spectra being supplied at the input of the neural network, and the second spectra serving to compute a cost function intended to adjust the weights and biases of the neural network; and
a prediction step, using the trained neural network, comprising:
supplying, at the input of the trained neural network, the neutron spectrum from photo-nuclear reactions induced by irradiating the material with said second photon source; and
if the irradiated material includes, in its composition, at least one chemical element chosen from carbon, nitrogen, oxygen or chlorine, obtaining, at the output of the trained neural network and for at least one specific energy of interest of said second photon source, a predicted neutron spectrum, including discrete neutron components from photo-nuclear reactions of said chemical element to be detected with said specific energy of interest.

2. Method according to claim 1, wherein the specific energy of interest is greater than or equal to 17 MeV.

3. Method for detecting a chemical element to be detected chosen from carbon, nitrogen, oxygen or chlorine, the method comprising:
irradiating, with a multi-energy photon source, a material comprising said chemical element;
detecting the neutrons emitted by the irradiated material and acquiring a corresponding neutron spectrum;
extracting, from the neutron spectrum acquired, discrete neutron components due to said chemical element to be detected, by implementing the method according to claim 1, the photon source being the first source;
comparing the discrete neutron components extracted with a library of discrete neutron components associated with carbon, nitrogen, oxygen and chlorine, and identifying a correlation, whereby the presence of said chemical element is inferred.

4. Method for detecting a material to be detected contained in an object to be probed, the material to be detected comprising at least one chemical element to be detected chosen from carbon, nitrogen, oxygen or chlorine, and the object to be probed comprising at least one other different chemical element, capable of emitting neurons and which is not carbon, nitrogen, oxygen or chlorine, the method comprising:
irradiating, with a photon source, the object to be probed and the material to be detected contained therein;
detecting the neutrons emitted by the object to be probed and the material to be detected and acquiring a corresponding neutron spectrum;
extracting, from the neutron spectrum acquired, discrete neutron components due to said at least one chemical element to be detected, by implementing the method according to claim 1, the photon source being the second source;
comparing the discrete neutron components extracted with a library of discrete neutron components associated with materials comprising said at least one first chemical element, and identifying a correlation, whereby the presence of said material to be detected is inferred.
